(19) United States
(12) Plant Patent Application Publication
(10) Pub. No.: US 2018/0168081 P1
(43) Pub. Date: Jun. 14, 2018

(54) HOP PLANT NAMED 'HBC 640'

(50) ;

(71) Applicant: HOP BREEDING COMPANY, L.L.C., Yakima, WA (US)

(72) Inventors: Eugene G. Probasco, Yakima, WA (US); Jason Perrault, Toppenish, WA (US)

(21) Appl. No.: 15/732,378

(22) Filed: Oct. 31, 2017

Related U.S. Application Data
(60) Provisional application No. 62/496,934, filed on Nov. 1, 2016.

(51) Int. Cl. A01H 5/02

(52) U.S. Cl. PLT/236

(57) ABSTRACT

A new hop plant named ‘HBC 640’ is disclosed. ‘HBC 640’ is used for its exceptional and unique aromatic qualities.

### CROSS-REFERENCE TO RELATED APPLICATIONS

Priority is claimed with respect to U.S. Provisional Patent Application 62/496,934 filed on Nov. 1, 2016.

### STATEMENT REGARDING FEDERALLY SPONSORED RESEARCH OR DEVELOPMENT

None

Genus and species: Humulus lupulus.

Variety denomination: ‘HBC 640’.

### BACKGROUND OF THE INVENTION

‘HBC 640’ is a product of a controlled breeding program carried out by the inventors in the Yakima Valley of Washington State. ‘HBC 640’ was one of several seedlings resulting from a controlled cross made in 2008 with female parent YCR 45 (unpatented) and male parent 20-9-10. Seedling plants from this cross were planted in 2009 and screened for disease resistance and sex in a greenhouse and field nursery near Granger, Wash. A single plant of ‘HBC 640’ was selected in 2012. In 2013 ‘HBC 640’ was expanded by asexual tissue culture propagation to 14 plants in an evaluation block near Toppenish, Wash. In 2015 ‘HBC 640’ was further expanded by asexual tissue culture propagation to a 1 acre test block near Toppenish, Wash. The ‘HBC 640’ plants have now been observed and evaluated for several years. Throughout several generations of asexual propagation, ‘HBC 640’ has been observed to retain its distinctive characteristics and remain true to type.

### BRIEF DESCRIPTION OF THE PHOTOGRAPHS

FIG. 1 illustrates a mature ‘HBC 640’ hop plant grown on a trellis;

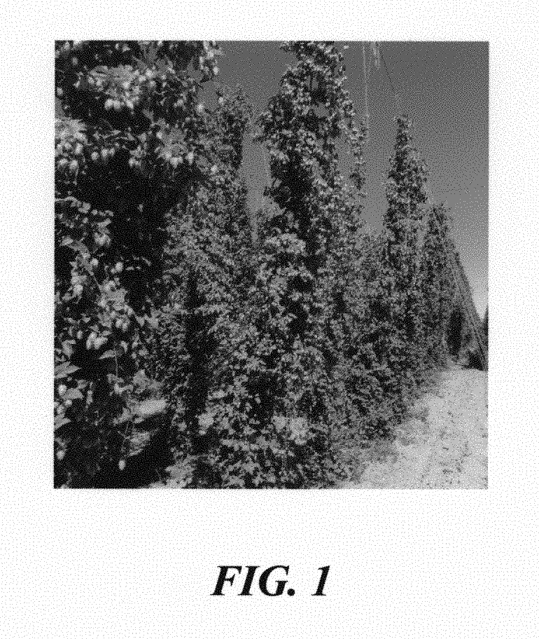

FIG. 2 illustrates whole cones and cross sections of cones of the ‘HBC 640’ hop plant;

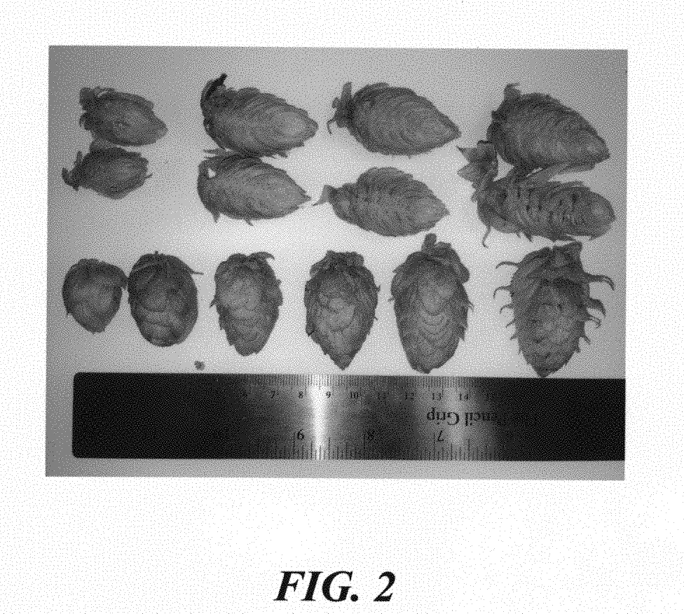

FIG. 3 illustrates the bine of a mature ‘HBC 640’ hop plant; and.

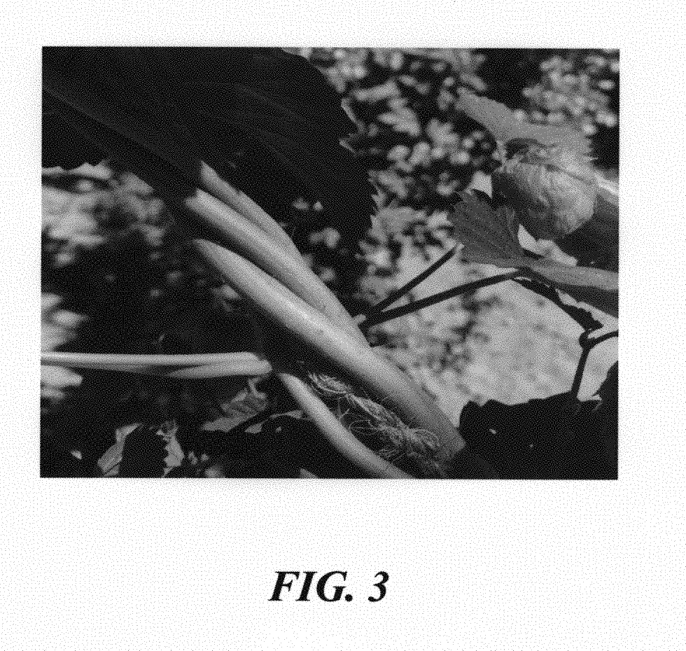

FIG. 4 illustrates cones on a mature ‘HBC 640’ hop plant.

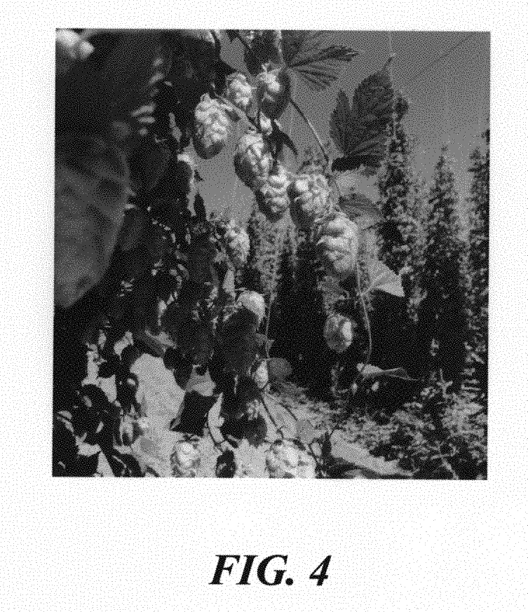

FIG. 5 illustrates leaves on a mature ‘HBC 640’ hop plant

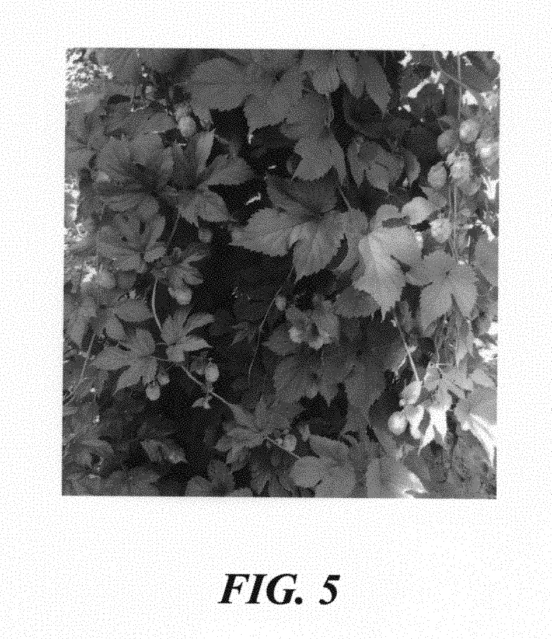

FIG. 6 illustrates leaves of a mature ‘HBC 640’ hop plant.

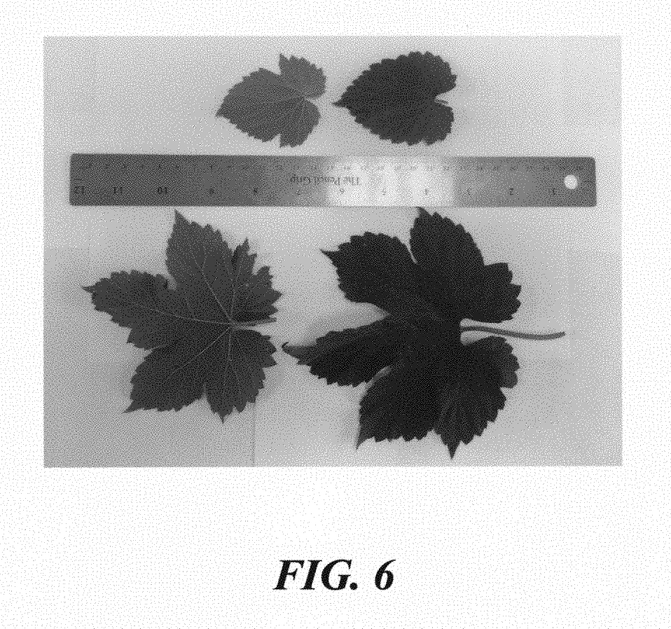

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2016 growing seasons at Toppenish, Wash. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. All color references are based on The Royal Horticultural Society Color Chart.
Ploidy: Diploid (2n=2x).
Sex: Female.
Use: Brewing beer and ale.
Harvest date: August 20 -30 (during 2015 to 2016 growing seasons at Toppenish, Wash.).
Yield: Average yield is 1800 to 2250 kg/hectare.
Disease reaction:
   Powdery mildew.—Moderate resistant.
   Downy mildew.—Unknown.
   Verticillium wilt.—Unknown.
Oil analytical characteristics (averages):
   % Alpha acid (% cone weight).—8.1%.
   % Beta acid (% cone weight).—3.2%.
   % Cohumulone (% alpha acids).—19.2%.
   Total oil (ml/100 g of cones).—1.4% Oil.
   Hop storage index.—<17.8%.
   Aroma profile.—Floral, citrus, and herbal spice.
   A-pinene.—0.07% Total Oil.
   B-pinene.—0.59% Total Oil.
   Myrcene.—41.05% Total Oil.
   2-methyl-butyl isobutyrate.—0.19% Total Oil.
   Limonene.—0.20% Total Oil.
   Linalool.—0.39% Total Oil.
   Caryophyllene.—11.53% Total Oil.
   Farnesene.—0.03% Total Oil.
   Humulene.—23.07% Total Oil.
   Citral.—0.40% Total Oil.
   Geranyl acetate 1.—0.23% Total Oil.
   Geranyl acetate 2.—0.03% Total Oil.
   Citronellol.—1.53% Total Oil.
   Nerol.—0.20% Total Oil.
   Geraniol.—0.95% Total Oil.
   B-lonene.—0.02% Total Oil.
   Carophyllene oxide.—0.24% Total Oil.
Bine:
   Color.—Green 143B.
   Stripe present.—Yes.
   Stripe color.—Greyed Purple 183D.
   Stipule direction.—Horizantal.
   Bine diameter.—8.0 mm at base; 11 mm at nine feet; 8.0 mm at terminal end of eighteen feet.
Leaf:
   Arrangement.—Opposite.
   Shape.—Palmately Lobed.
   Average length of mature leaf.—13.5 cm.
   Average width of mature leaf.—14.0 cm.
   Color of mature leaf upper surface.—Yellow-Green 147A.
   Color of mature leaf lower surface.—Yellow-Green 147B.
   Color of immature leaf upper surface.—Green 143A.
   Color of immature leaf lower surface.—Yellow-Green 146B.
   Number of lobes.—1, 3 or 5.
   Margin.—Serrate.
   Serrations per inch.—5-7.
   Average petiole length (mature)—52 mm.
   Petiole color at base.—Grey Purple 183B.
   Venation.—Palmate.
   Vein color.—Yellow-Green 144B.
Cone:
   Avg. length.—46 mm.
   Avg. diameter.—25 mm.
   Bract tip color.—Yellow-Green 143A.
   Bract base color.—Yellow-Green 145A.
   Bracteole color.—Yellow-Green 145B.
   Cone shape.—Ovate.
   Bract shape.—Deltoid.
   Bract tip shape.—Acuminate — Mucronate.
   Bract tip position.—Loosely appressed — Recurved.
   Bracteole shape.—Rotund.
   Pickability.—Good.
   Lupulin glands color.—Yellow 2B.

1. A new and distinct Hop plant as illustrated and described herein.
